(19) United States
(12) Patent Application Publication
(10) Pub. No.: US 2021/0251906 A1
(43) Pub. Date: Aug. 19, 2021

(54) PREGABALIN EXTENDED-RELEASE FORMULATIONS

(71) Applicant: Mylan Inc., Canonsburg, PA (US)

(72) Inventors: Timothy M. Long, Canonsburg, PA (US); John Kirsch, Morgantown, WV (US); Joseph Morott, Morgantown, WV (US); Grant Heinicke, Morgantown, WV (US)

(73) Assignee: Mylan Inc., Canonsburg, PA (US)

(21) Appl. No.: 17/255,545

(22) PCT Filed: Jun. 28, 2019
(86) PCT No.: PCT/US2019/039752

§ 371 Date: Dec. 23, 2020

Related U.S. Application Data
(60) Provisional application No. 62/691,377, filed on Jun. 28, 2018.

(51) Int. Cl. A61K 9/20; A61K 31/197

(52) U.S. Cl. CPC A61K 9/2027; A61K 31/197; A61K 9/2031; A61K 9/2054

(57) ABSTRACT

Extended-release formulations can be prepared that comprise a core and an optional coating layer formed over the core. The core comprises a therapeutically effective amount an active pharmaceutical ingredient (API), a non-swelling matrix forming agent comprising a water-soluble agent and a water-insoluble polymer; one or more extended-release agents; an optional, wicking agent; and one or more optional excipients. Such formulations may be useful for preparing extended-release formulations of pregabalin that are suitable for once-daily dosing for the treatment of neuropathic pain associated with diabetic peripheral neuropathy (DPN) or postherpetic neuralgia (PHN).

### CROSS-REFERENCE TO RELATED APPLICATIONS

This application claims priority to and the benefit of the filing date of U.S. Provisional Application Ser. No. 62/691,377, filed on Jun. 28, 2018, the contents of which are hereby incorporated by reference in its entirety.

### FIELD OF THE INVENTION

The present invention relates to extended-release formulations, processes for preparing the same, and uses of the formulations for treatment and/or management of disease.

### BACKGROUND OF THE INVENTION

Pregabalin or (S)-3-(aminomethyl)-5-methylhexanoic acid is an analog of gamma-aminobutyric acid (GABA) that decreases central neuronal excitability by binding to an auxiliary alpha-2-delta subunit of a voltage-gated calcium channel on neurons in the central nervous system.

Pregabalin, disclosed in U.S. Pat. Nos. 5,563,175 and 6,197,819, marketed under the name LYRICA® in the U.S is used in the treatment of peripheral neuropathic pain, epilepsy and generalized anxiety disorder. Pregabalin is also effective at treating chronic pain in disorders such as fibromyalgia and spinal cord injury. U.S. Pat. No. 6,117,906 discloses the use of pregabalin in treating anxiety; U.S. Pat. No. 6,001,876 discloses the use of pregabalin in treating pain; U.S. Pat. No. 6,127,418 discloses the use of pregabalin in treating gastrointestinal damage. PCT Publication W098/58641 discloses use of pregabalin as an anti-inflammatory agent. Pregabalin is freely soluble in water and in basic and acidic aqueous solutions. Elimination half-life of pregabalin is about 6.3 hours. It is available as an immediate release formulation in capsules and is administered two- or three-times daily.

### SUMMARY OF THE INVENTION

After extensive research, extended release dosage forms of pregabalin in one or more release rate extending agents and a diluent are provided. Such may provide an extended-release formulation (e.g., tablet) that offers improved control and reliability while retaining the ability to control drug release.

In one aspect, the present disclosure provides extended-release formulations comprising a core and an optional coating layer formed over the core, wherein the core comprises or consists essentially of: a therapeutically effective amount an active pharmaceutical ingredient (API); a non-swelling matrix forming agent comprising a water-soluble agent and a water-insoluble polymer; one or more extended-release agents; an optional, wicking agent; and one or more optional excipients.

In another aspect, the present disclosure provides modified release tablets comprising the formulation of the preceding aspect.

In another aspect, the present disclosure provides processes for preparing formulations of the preceding aspects, for example, comprising combining an API, a matrix forming agent; at least two extended-release agents; and an optional, wicking agent to provide a first blend; and combining the first blend with one or more optional excipients to provide a final blend.

Another aspect of the invention provides a method of treating a condition or disorder in a subject that is responsive to pregabalin, the method comprising orally administering to the subject a pharmaceutical composition, described herein, once daily. “Treating” generally refers to reversing, alleviating, inhibiting the progress of, or preventing a disorder or condition in a subject, or to preventing one or more symptoms of such disorder or condition in the subject. “Treatment” refers to the act of “treating” as defined immediately above. In particular, such formulations may be suitable for once-daily dosing for the treatment of neuropathic pain associated with diabetic peripheral neuropathy (DPN) or treatment of postherpetic neuralgia (PHN) (e.g., by improvement in pain intensity from baseline). Such formulations may also be suitable for once-daily dosing for treatment of seizure disorders (e.g., epilepsy), anxiety, alcohol use disorder, fibromyalgia, cancer pain (e.g., mucositis pain in patients undergoing chemoradiation therapy), post-operative pain, restless legs syndrome, and nerve pain due to spinal cord injury.

Surprisingly, drug release from tablets of equal size containing different amounts of pregabalin occurs at the same rate in a variety of aqueous dissolution media: Matrix tablets of equal size, made with either soluble or insoluble polymers or mixtures thereof, usually release slower as the drug content decreases because of the lower concentration gradient through the tablet compared to higher strengths and the higher amount of polymer or filler that the drug has to traverse.

Also unexpected was equivalent in vivo performance without a significant increase in physical dimensions after exposure to aqueous media. None of the materials used in the disclosed compositions are generally regarded as swelling agents that increase tablet dimensions to the extent of gastric retention.

In certain embodiments, the formulations herein may have a reduced or absent food effect when dosed to a person in need following a meal.

### BRIEF DESCRIPTION OF THE DRAWINGS

FIG. 1 is a graph showing % release of pregabalin API from the formulation of Example 1 (330 mg) as compared to Lyrica® CR (330 mg) using the US FDA Method (0.06N HCl, App. II @ 50 rpm); (X & broken line=Lyrica CR, 330 mg; open squares & solid line=Example 1, 330 mg).

FIG. 2 is a graph showing % release of pregabalin API from the formulation of Example 1 (330 mg) as compared to Lyrica® CR (330 mg) in a pH 4.5 acetate buffer, App II @ 50 rpm (X & broken line=Lyrica CR, 330 mg; open squares & solid line=Example 1, 330 mg).

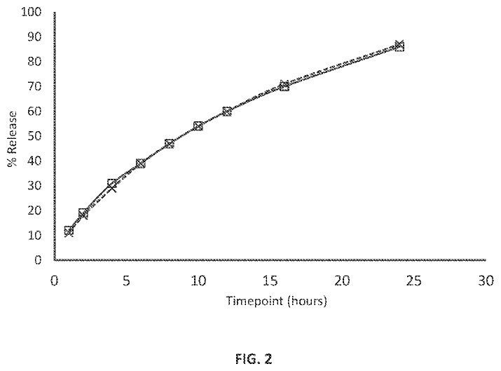

FIG. 3 is a graph showing % release of pregabalin API from the formulation of Example 2 (165 mg) as compared to Lyrica® CR (165 mg) in a pH 4.5 acetate buffer, App II @ 50 rpm (X & broken line=Lyrica CR, 165 mg; open squares & solid line=Example 2, 165 mg).

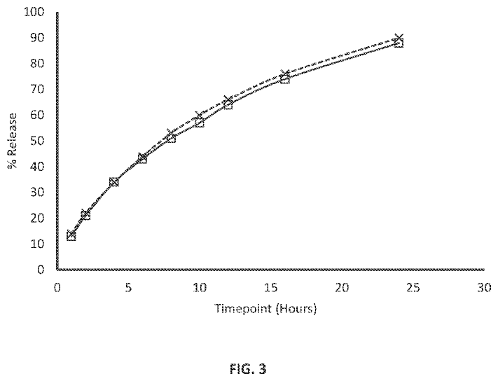

FIG. 4 is a graph showing % release of pregabalin API from the formulation of Example 3 (82.5 mg) as compared to Lyrica® CR (82.5 mg) in a pH 4.5 acetate buffer, App II @ 50 rpm (X & broken line=Lyrica CR, 82.5 mg; open squares & solid line=Example 3, 82.5 mg).

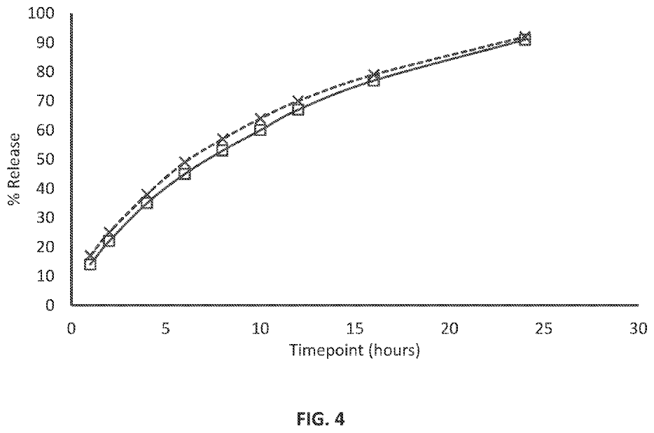

FIG. 5 is a graph showing % release of pregabalin API from the formulation of Example 4 (330 mg) as compared to Lyrica® CR (330 mg) in water, App II @ 150 rpm (+=Lyrica CR, 330 mg; solid circle=Example 4, 330 mg).

FIG. 6 is a graph showing % release of pregabalin API from the formulation of Example 5 (165 mg) as compared to Lyrica® CR (165 mg) in water, App II @ 150 rpm (+=Lyrica CR, 165 mg; solid square=Example 5, 165 mg).

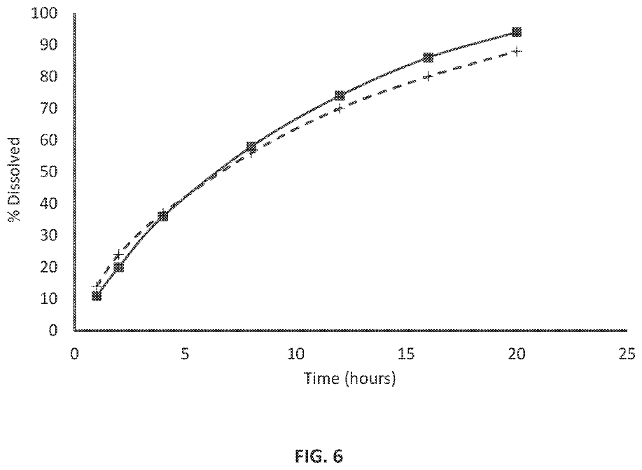

FIG. 7 is a graph showing % release of pregabalin API from the formulation of Example 6 (82.5 mg) as compared to Lyrica® CR (82.5 mg) in water, App II @ 150 rpm (+=Lyrica CR, 82.5 mg; solid triangle=Example 6, 82.5 mg).

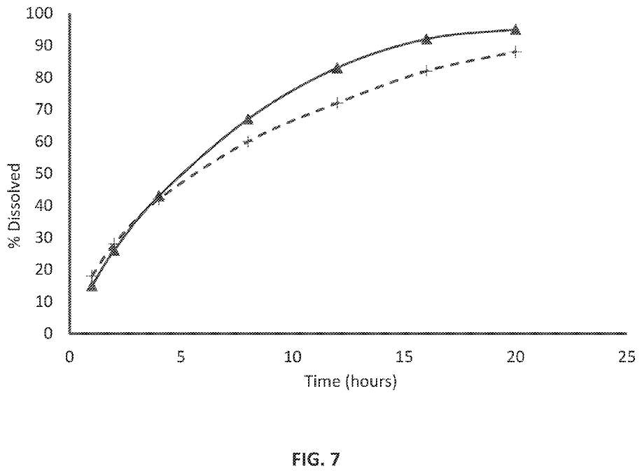

FIG. 8 is a graph showing % release of pregabalin API from the formulation of Example 7 (330 mg) as compared to Lyrica® CR (330 mg) in water, App II @ 150 rpm (x=Lyrica CR, 82.5 mg; open diamonds=Example 7, 330 mg).

### DETAILED DESCRIPTION OF THE INVENTION

The pharmaceutical dosage forms described herein, after oral administration, release an active pharmaceutical ingredient (API), such as pregabalin, in a sustained manner. After rigorous experimentation, the extended release dosage forms of APIs (e.g. pregabalin) can provide in-vitro dissolution profiles that are suitable for once a day administration and are bioequivalent to the corresponding dosage strength of Lyrica® CR (Pfizer, New York, N.Y.).

Accordingly, in one aspect, the present disclosure provides extended-release formulations comprising a core and an optional coating layer surrounding the core, wherein the core comprises or consists essentially of a therapeutically effective amount an active pharmaceutical ingredient (API); a non-swelling matrix forming agent comprising a water-soluble agent and a water-insoluble polymer; one or more extended-release agents; an optional, wicking agent; and one or more optional excipients.

The terms “extended release” or “controlled release” or “sustained release” or “modified release” mean that the referenced dosage form, when tested according to USP 711 in a Type-I basket apparatus operating at 100 rpm in a medium of 0.06 N HCl (900 mL), release the active ingredient over an extended period of time, for example from at least about 2 hours to about 24 hours; or from at least about 2 hours to about 20 hours; or from at least about 4 hours to about 16 hours; or from at least about 4 hours to about 12 hours etc. Notably, “extended release” or “controlled release” or “sustained release” or “modified release” exclude immediate release dosage forms whose in vitro dissolution process requires no more than 60 min.

The terms “dosage form” or “composition” or “formulation” refer to pharmaceutical compositions that are suitable for oral administration to a human subject, including, but not limited to oral formulation such as tablets, capsules, powders, granules, pellets, beads, and minitablets.

The formulation typically comprises a therapeutically effective amount of the active pharmaceutical ingredient (API), such as, pregabalin or a pharmaceutically acceptable salt or solvate thereof. “Therapeutically effective amount” of an API refers to the quantity that may be used for treating a subject (i.e., a mammal, including a human) and is generally in the range of about 0.001 to about 100 mg/kg/day for an adult, and is often in the range of about 0.1 to about 50 mg/kg/day for an adult. For an adult human, a typical daily dose of a drug is in the range of about 1 mg to about 1000 mg. In one example, the daily dose of pregabalin for an adult human may be in the range of about 50 mg to about 1800 mg and is often in the range of about 50 mg to about 900 mg.

The API can be selected from the group consisting of atagabalin, baclofen, gabapentin enacarbil, gabapentin, mirogabalin, pregabalin, valnoctamide, valproate pivoxil, valproic acid, valpromide, vigabatrin, (1S,3S)-3-amino-4-(difluoromethylene)-cyclopentanecarboxylic acid (CPP-115), (1α,3α,5α)-3-(aminomethyl)-bicyclo[3.2.0]heptane-3-acetic acid (PD-217,014), (2S,3S,4S)-α-(carboxycyclopropyl)glycine (L-CCG-I), 4-amino-5-hexynoic acid (MDL 71645), 4-amino-5-fluoropentanoic acid (MDL 71109), 4-methylpregabalin, and pharmaceutically acceptable salts thereof. In certain embodiments, the API comprises pregabalin or a pharmaceutically acceptable salt or solvate thereof.

The amount of API (e.g., pregabalin) in the core of the extended-release formulations is from 5% (e.g., 70 mg API in a 1400 mg tablet gives 5%) to 45% (e.g., 350 mg API in a 780 mg tablet) by weight of the core; or from about 6% (e.g., 75 mg in 1250 mg tablet) to 40 wt % (e.g., 350 mg in 875 mg tablet); or from about 7-38 wt % of the core; or from about 7.17% (e.g., 82.5 mg API in 1150 mg tablet) to 36.67% (e.g., 330 mg in 900 mg tablet) by weight of the core; or from about 5-40 wt % of the core; or from about 5-35 wt % of the core; or from about 5-30 wt % of the core.

“About” as used herein means +/−10% of the referenced value; or +/−5% of the referenced value; or +/−2% of the referenced value.

In certain embodiments, the API is pregabalin and is present in an amount from about 50 mg to about 500 mg; or from about 50 mg to about 400 mg; or from about 75 mg to about 400 mg; or from about 80 mg to about 350 mg; or in an amount selected from 50 mg, 75 mg, 80 mg, 82.5 mg, 85 mg, 90 mg, 100 mg, 110 mg, 120 mg, 125 mg, 130 mg, 140 mg, 150 mg, 175 mg, 200 mg, 225 mg 250 mg, 300 mg, 325 mg, 330 mg, 335 mg, and 350 mg. In certain embodiments, pregabalin is present in an amount of about 82.5 mg, 165 mg, or 330 mg.

Pregabalin can be any pharmaceutically acceptable form, including its free form (zwitterion), any salt, polymorphic form, solvated form (including any hydrate), and/or single enantiomer (e.g., S-pregabalin) or mixture of enantiomers (e.g. racemic mixtures). Suitable forms include those disclosed in, for example, U.S. Pat. No. 5,637,767 (crystalline monohydrate); U.S. Pat. No. 7,417,165 (crystalline hemihydrate); and U.S. Patent Application Publication Nos. 20060270871 (anhydrous crystalline “Form I”) and 20080014280 (amorphous). When a salt, solvate, and/or hydrate of pregabalin is present, then the preceding masses refer to the equivalent amount of unsolvated pregabalin free-base (i.e., (S)-3-(aminomethyl)-5-methylhexanoic acid). Pregabalin salts include, without limitation, acid addition salts and base addition salts, including hemisalts. Pharmaceutically acceptable acid addition salts may include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Potentially useful salts include acetate, aspartate, benzoate, chlorobenzoate, methyl benzoate, dinitrobenzoate, besylate, bicarbonate, carbonate, bisulfate, sulfate, pyrosulfate, bisulfite, sulfite, borate, camsylate, caprylate, citrate, edisylate, esylate, formate, fumarate, gluceptate, gluconate, glucuronate, hexafluorophosphate, hibenzate, hydrochloride, chloride, hydrobromide, bromide, hydroiodide, iodide, isethionate, isobutyrate, lactate, malate, maleate, malonate, mandelate, mesylate, methylsulfate, naphthylate, 2-napsylate, nicotinate, nitrate, orotate, oxalate, palmitate, pamoate, phosphate, hydrogen phosphate, dihydrogen phosphate, metaphosphate, pyrophosphate, phthalate, propionate, saccharate, sebacate, stearate, suberate, succinate, tartrate, tosylate, trifluoroacetate, and the like.

The extended release formulations can provide steady-state pharmacokinetics for once-daily administration of an extended-release formulation as described herein following an evening meal and comprising about 165 mg of pregabalin of one or more of the following values:

| | |
|---|---|
| Cₘₐₓ (μg/mL) | between about 1.0 and 4.0 |
| Tₘₐₓ (h) | between about 5.0 and 12.0 |
| AUC₂₄ (μg•h/mL) | between about 15 and 35 |
| Cₘᵢₙ (μg/mL) | between about 0.2 and 0.8 |

where AUC₂₄ (the area under the curve over 24 hours), Cmax (peak concentrations), Cmin (minimum concentration) are reported as the geometric mean (% CV); and Tmax (time to peak concentration) is reported as a median (range). Steady-state pharmacokinetics for once-daily administration of lower (e.g., 82.5 mg) and higher (e.g., 330 mg) extended release formulations can show dose-proportional increases in maximum plasma concentration (Cₘₐₓ) and area under the plasma concentration-time curve (AUC) values noted above.

In other embodiments, the pregabalin extended release formulations provide steady-state pharmacokinetics for once-daily administration of an extended-release formulation as described herein and comprising about 165 mg of pregabalin of one or more of the following values:

| | |
|---|---|
| Cₘₐₓ (μg/mL) | between about 1.5 and 2.5 |
| Tₘₐₓ (h) | between about 6.0 and 10.0 |
| AUC₂₄ (μg•h/mL) | between about 25 and 35 |
| Cₘᵢₙ (μg/mL) | between about 0.3 and 0.7 |

Steady-state pharmacokinetics for once-daily administration of lower (e.g., 82.5 mg) and higher (e.g., 330 mg) extended release formulations can show dose-proportional increases in maximum plasma concentration (Cₘₐₓ) and area under the plasma concentration-time curve (AUC) values noted above.

In other embodiments, the pregabalin extended release formulations provide steady-state pharmacokinetics for once-daily administration of an extended-release formulation as described herein and comprising about 165 mg of pregabalin of one or more of the following values:

| | |
|---|---|
| Cₘₐₓ (μg/mL) | between about 1.8 and 2.2 |
| Tₘₐₓ (h) | between about 7.0 and 9.0 |
| AUC₂₄ (μg•h/mL) | between about 27 and 33 |
| Cₘᵢₙ (μg/mL) | between about 0.4 and 0.6 |

Steady-state pharmacokinetics for once-daily administration of lower (e.g., 82.5 mg) and higher (e.g., 330 mg) extended release formulations can show dose-proportional increases in maximum plasma concentration (Cₘₐₓ) and area under the plasma concentration-time curve (AUC) values noted above.

Core

As noted above, the core of the extended-release compositions herein can comprise or consists essentially of a therapeutically effective amount an active pharmaceutical ingredient (API); a non-swelling matrix forming agent comprising a water-soluble agent and a water-insoluble polymer; one or more extended-release agents; an optional, wicking agent; and one or more optional excipients.

The term “matrix forming agent” as used herein means pharmaceutically acceptable materials suitable for use in preparing oral dosage forms that imparts structural integrity and helps control or extend the rate of drug (e.g. API) release, among other functions.

The term “swelling” as used herein means the referenced material can absorb water from the gastric fluid which causes the solid dosage form to expand in size, and may also influence the drug release rate by, for example, creating channels. The term “non-swelling” as used herein means the referenced material (e.g., polymer or disintegrant) may absorb water from gastric fluid but does not substantially expand in size.

Herein, the matrix forming agent can comprise two components, a water-soluble agent and a water-insoluble polymer. The term “water-soluble” as used herein means the referenced material has an aqueous solubility of greater than 1 mg/mL when at room temperature (about 23° C.). The term “water-insoluble” as used herein means the referenced material has an aqueous solubility of less than 1 mg/mL at room temperature (about 23° C.).

In certain embodiments, the core can comprise about 10-85 wt % of the matrix forming agent. In another embodiment, the core can comprise about 15-75 wt % of the matrix forming agent. In another embodiment, the core can comprise about 15-50 wt % of the matrix forming agent. In another embodiment, the core can comprise about 20-50 wt % of the matrix forming agent. In another embodiment, the core can comprise about 20-40 wt % of the matrix forming agent. In another embodiment, the core can comprise about 20-30 wt % of the matrix forming agent; or about 25-35 wt % of the matrix forming agent; or about 25-30 wt % of the matrix forming agent; or about 30-50 wt % of the matrix forming agent; or about 30-45 wt % of the matrix forming agent.

Examples of suitable water-soluble agents include, but are not limited to a poly(vinyl pyrrolidone), a polyethylene glycol, a salt, a sugar, a sugar alcohol, an amino acid, or a mixture thereof.

“Polyethylene glycol” as used herein, refers to a low-molecular weight (M_{w}) polymer having a weight-averaged molecular weights of 200 Da up to about 25 kDa, that can be produced by base-catalyzed ring-opening polymerization of ethylene oxide. For example, the reaction can be initiated by adding ethylene oxide to ethylene glycol, with potassium hydroxide as catalyst. Examples of commercial polyethylene glycols include those from Polysciences, Inc. (Warrington, Pa.) such as PEG400 (M_{w} about 400 Da), PEG3400 (M_{w} about 3400 Da), PEG7500 (M_{w} about 7500 Da), and PEG10K-16 K (M_{w} about 10 kDa-16 kDa).

Suitable salts include, but are not limited to alkali, alkaline earth, or ammonium carbonates, bicarbonates, or halides, such as sodium chloride, sodium carbonate, sodium bicarbonate, calcium carbonate, ammonium bicarbonate, and mixtures thereof. Suitable sugars include, but are not limited to, mono- and di-saccharides such as glucose, fructose, galactose, lactose, maltose, sucrose, and mixtures thereof. Suitable sugar alcohols include, but are not limited to, xylitol, sorbitol, and mixtures thereof. Suitable amino acids include, but are not limited to, natural and synthetic alpha-amino acids such as glycine, alanine, leucine, isoleucine, valine, proline, lysine, arginine, aspartic acid, glutamic acid, asparagine, glutamine, cysteine, methionine, tyrosine, tryptophan, histidine, phenylalanine, serine, threonine, each in D-, L-, or racemic forms, and mixtures thereof.

In certain embodiments, the water-soluble agent is a poly(vinyl pyrrolidone) or a polyethylene glycol. In another embodiment, the water-soluble agent is a poly(vinyl pyrrolidone). In certain embodiments, the water-soluble agent is a poly(vinyl pyrrolidone) having an Mw (weight-averaged molecular weight) between about 1 kDa and 100 kDa; or between about 1 kDa and 75 kDa; between about 2 kDa and 75 kDa; or between about 2 kDa and 60 kDa. Examples of commercial poly(vinyl pyrrolidone) include those from BASF SE (Lampertheim, Germany), such as, Kollidon®-branded products, Kollidon® 12 (M_{w} about 2-3 kDa), Kollidon® 17 (M_{w} about 7-11 kDa), Kollidon® 25 (M_{w} about 28-34 kDa), and Kollidon® 30 (M_{w} about 44-54 kDa).

Examples of suitable water-insoluble polymers include, but are not limited to a poly(vinyl acetate), a polyacrylate alkyl ester, a polylactide, a polyglycolide, a poly(lactide-co-glycolide), cellulose acetate, an alkyl cellulose, a polyacrylic acid (e.g., a CARBOPOL), or a mixture thereof.

Polyacrylate alkyl ester refers to the polyester formed by radical-catalyzed polymerization of C1-C6 alkyl esters of acrylic acid or methacrylic acid. Examples include, but are not limited to, poly(iso-propyl methacrylate), poly(methyl methacrylate), poly(tert-butyl methacrylate), poly(n-butyl methacrylate), poly(iso-propyl acrylate), poly(methyl acrylate), poly(tert-butyl acrylate), poly(n-butyl acrylate) and mixtures thereof. Polylactides include poly(L-lactic acid), poly (D-lactic acid), poly(D,L-lactic acid), and mixtures thereof. Polyglycolide refers to the polyester formed by, for example, dehydration of glycolic acid. Poly(lactide-co-glycolide) refers to co-polyesters of L-lactic acid, D-lactic acid, D,L-lactic acid, or a mixture thereof, with glycolic acid. Such co-polymers may be random or block co-polymers.

“Alkylcellulose” means a cellulose polymer having a least a portion of the glucose hydroxyl groups substituted with only C₁-C₆alkyl groups. Examples of alkylcelluloses as used herein include, but are not limited to, methylcellulose and ethylcellulose. The term “C₁-C₆alkyl” as used herein means an univalent group derived from a linear or branched alkane by removal of a hydrogen atom from any carbon atom, e.g., —CₙH₂ₙ₊₁, wherein n is an integer selected from 1 to 6. Examples of C₁-C₆alkyl groups include, but are not limited to, methyl, ethyl, n-propyl, i-propyl, n-butyl, i-butyl, n-pentyl, n-hexyl. The term “hydroxyC₁-C₆alkyl” as used herein means a C₁-C₆alkyl substituted with one or more hydroxy (—OH) functional groups. Examples of hydroxyC₁-C₆alkyl groups include, but are not limited to, hydroxymethyl, 2-hydroxyethyl, 2-hydroxypropyl, 3-hydroxypropyl, 4-hydroxybutyl, 5-hydroxypentyl, and 6-hydroxyhexyl.

For example, the alkylcellulose can be selected from the group consisting of an ethylcellulose, a methylcellulose, and mixtures thereof. Suitable commercially available ethycelluloses include, but are not limited to, ETHOCEL 4 Premium, ETHOCEL 7 Premium, ETHOCEL 7FP Premium, ETHOCEL 10 Premium, ETHOCEL 10FP Premium, ETHOCEL 20 Premium, ETHOCEL 45 Premium, ETHOCEL 100 Premium, and ETHOCEL 100FP Premium (ETHOCEL product from Dow Chemical, Midland, Mich.; each having an ethoxyl content of about 48.0-49.5 wt %). In one example, the ethylcellulose comprises ETHOCEL 10FP Premium.

In certain embodiments, the water-insoluble polymer is a poly(vinyl acetate), cellulose acetate, ethyl cellulose, a polyacrylic acid, or a mixture thereof. In certain other embodiments, the water-insoluble polymer is a poly(vinyl acetate), cellulose acetate, ethyl cellulose, or a mixture thereof. In certain embodiments, the water-insoluble polymer is a poly(vinyl acetate).

In another embodiment, the matrix forming agent comprises, a water-soluble agent that is a poly(vinyl pyrrolidone) or a polyethylene glycol, or a mixture thereof; and a water-insoluble polymer that is a poly(vinyl acetate), a polyacrylic acid, cellulose acetate, ethyl cellulose, or a mixture thereof.

In another embodiment, the matrix forming agent comprises, a water-soluble agent that is a poly(vinyl pyrrolidone) or a polyethylene glycol, or a mixture thereof; and a water-insoluble polymer that is a poly(vinyl acetate), cellulose acetate, ethyl cellulose, or a mixture thereof.

In another embodiment, the matrix forming agent comprises poly(vinyl pyrrolidone) and poly(vinyl acetate). One suitable commercially available matrix forming agent comprising poly(vinyl pyrrolidone) and poly(vinyl acetate) is KOLLIDON® SR (BASF SE, Lampertheim, Germany) which is nominally an 80/19 (w/w) mixture of mixture of poly(vinyl acetate) and poly(vinyl pyrrolidone), respectively (the remainder being 0.8% sodium lauryl sulfate and 0.2% silica). The weight-averaged molecular weight (M_{w}) of the polyvinyl acetate is about 450,000 Da and the weight-averaged molecular weights (M_{w}) of the poly(vinyl pyrrolidone) is about 50,000 Da.

The term “extended-release agent” as used herein means a pharmaceutically acceptable material suitable for use in preparing oral dosage forms that control or extend the rate of drug (e.g., API) release. The extended-release agent in the present formulations can comprise a non-ionic polymer and/or an ionic polymer. The term “ionic polymer” as used herein means the referenced polymer contains functional groups having a pKa less than 14. The term “non-ionic polymer” as used herein means the referenced polymer does not contain functional groups having a pKa less than 14.

Examples of suitable ionic polymers include, but are not limited to, a poly(acrylic acid), a carbomer, alginic acid, carrageenan, xanthan gum, carrageenan, or mixtures thereof.

“Carbomers” as used herein refers to both carbomer homopolymer and carbomer copolymers. “Carbomer homopolymers” are high molecular weight poly(acrylic acid) cross-linked with allyl ethers of polyalcohols (such as pentaerythritol and sucrose). Examples of carbomer homopolymers include, but are not limited to, carbomer 941 (poly(acrylic acid) cross-linked with allyl ethers of pentaerythritol); and carbomer 934 (poly(acrylic acid) cross-linked with allyl ethers of sucrose). “Carbomer Copolymers” are high molecular weight copolymers of acrylic acid and a long-chain alkyl methacrylate cross-linked with allyl ethers of polyalcohols. Examples of carbomer copolymers include, but are not limited to, carbomer 1342 (copolymer of acrylic acid and a long-chain alkyl methacrylate cross-linked with allyl ethers of pentaerythritol). Commercially available carbomers include, but are not limited to CARBOPOL® 71G N (Lubriuzol Adv. Mat., Cleveland, Ohio), which meets United States Pharmacopeia/National Formulary (USP/NF) monograph for Carbomer Homopolymer Type A.

Examples of suitable non-ionic polymers include a polyethylene oxide, a polysaccharide (e.g., guar gum, inulin), a hydroxyalkyl alkylcellulose, or a mixture thereof.

Poly(ethylene oxide), also referred to herein as “polyethylene oxide” and “PEO,” is a linear polymer of unsubstituted ethylene oxide. Few examples of poly(ethylene oxide)s that are commercially available include, but are not limited to, POLYOX® and POLYOX® NF PEO products (Dow Chemical, Midland, Mich.), available in grades such as: WSR-205 (Mᵥ 600,000 Da); WSR-1105 (Mᵥ 900 kDa); WSR N-12K (Mᵥ 1 MDa); WSR N-60K (Mᵥ 2 MDa); WSR-301 (Mᵥ 4 MDa); WSR Coagulant (Mᵥ 5 MDa); WSR-303 (Mᵥ 7 MDa); and WSR-308 (Mᵥ 8 MDa). In one embodiment, the PEO has a viscosity-averaged molecular weight (Mᵥ) between about 1.0 MDa and about 8.0 MDa. In another embodiment, the PEO has a viscosity-averaged molecular weight (Mᵥ) between about 1.0 MDa and about 4.0 MDa. In another embodiment, the PEO has a viscosity-averaged molecular weight (Mᵥ) about 2.0 MDa, such as POLYOX® WSR N-60K (Mᵥ 2 MDa). “Mᵥ” refers to the viscosity average of the molecular weight.

“Hydroxyalkyl alkylcellulose” means a cellulose polymer having a least a portion of the glucose hydroxyl groups substituted with hydroxyC₁-C₆alkyl groups and another portion of the glucose hydroxyl groups substituted with C₁-C₆alkyl groups. Examples of hydroxyalkyl alkylcelluloses as used herein include, but are not limited to, hydroxyethyl methylcellulose (e.g., 2-hydroxyethyl methylcellulose) and hydroxypropyl methylcellulose (e.g., 2-hydroxypropyl methylcellulose). In certain embodiments, the hydroxyalkyl alkylcellulose is a hydroxypropyl methylcellulose.

In certain embodiments, the extended-release agent comprises a poly(acrylic acid) or a carbomer and a polyethylene oxide. In another embodiment, the extended-release agent comprises a carbomer and a polyethylene oxide.

The core can comprise about 10-85 wt % of the extended-release agents; or about 15-75 wt % of the extended-release agents; or about 15-50 wt % of the extended-release agents. In other embodiments, the core can comprise about 20-50 wt % of the extended-release agents; or about 20-40 wt % of the extended-release agents; or about 25-40 wt % of the extended-release agents; or about 25-35 wt % of the extended-release agents; or about 30-50 wt % of the extended-release agents; or about 30-60 wt % of the extended-release agents.

When the core contains the wicking agent (e.g., about 5-45 wt % or 15-30 wt % of the wicking agent), then the core can comprise about 50-90 wt % of the sum of the matrix forming agent and extended-release agents; or about 50-75 wt % of the sum of the matrix forming agent and extended-release agents; or about 50-70 wt % of the sum of the matrix forming agent and extended-release agents; or about 50-65 wt % of the sum of the matrix forming agent and extended-release agents.

When the core does not contain the wicking agent then the core can comprise about 55-95 wt % of the sum of the matrix forming agent and extended-release agents; or about 60-94 wt % of the sum of the matrix forming agent and extended-release agents; or about 62-93 wt % of the sum of the matrix forming agent and extended-release agents. In yet other embodiments, when the core does not contain the wicking agent, then the core can comprise about 55-95 wt % of the sum of the matrix forming agent and extended-release agents; or about 55-90 wt % of the sum of the matrix forming agent and extended-release agents.

In certain examples, the core can comprise about 53.6% (e.g., Example 1) up to 90.4 wt % (e.g., Example 6) of the sum of the matrix forming agent and extended-release agents.

The term “wicking agent” as used herein means a material with the ability to draw water into a matrix, for example, through capillary action. A wicking agent can do this with or without swelling. Examples of wicking agents that may be used include, but are not limited to, a microcrystalline cellulose, a powdered cellulose, magnesium aluminum silicate, sodium lauryl sulfate, a starch, a low-molecular weight polyvinylpyrrolidone, a clay (e.g., kaolin or bentonite), silicified microcrystalline cellulose (e.g., Prosolv®), alumina, or a mixture thereof.

In certain embodiments, the wicking agent is a non-swelling wicking agent. Examples of non-swelling wicking agent, include, microcrystalline cellulose, sodium lauryl sulfate, colloidal silicon dioxide, and low molecular weight polyvinylpyrrolidone.

In one embodiment, the wicking agent is a microcrystalline cellulose. Examples of suitable commercially available microcrystalline celluloses includes Avicel® PH 101, Avicel® PH 102, Avicel® PH 112, Avicel® PH 200, Avicel® PH 301, and Avicel® PH 302 (Avicel® brand products from FMC Corp, Philadelphia, Pa.). In certain embodiments, the wicking agent is a Avicel® PH 101.

In certain embodiments, the core can comprise about 0 wt % to about 45 wt % of the wicking agent. In another embodiment, the core can comprise about 1 wt % to about 45 wt % of the wicking agent. In another embodiment, the core can comprise about 5 wt % to about 45 wt % of the wicking agent. In another embodiment, the core can comprise about 15 to about 35 wt % of the wicking agent. In another embodiment, the core can comprise about 17.17 wt % (e.g., 330 mg in a 1150 mg tablet) to 29.83 wt % (e.g., 82.5 in a 1150 mg tablet) of the wicking agent.

In embodiments where the core does not contain a wicking agent, the core may comprise two or more extended release agents, such as, but not limited to, two or three extended release agents. For example, the core can comprise three extended release agents comprising a first non-ionic polymer and a first ionic polymer, each as defined above, and a third polymer. The third polymer can be a second non-ionic polymer or a second ionic polymer. In one example, the third polymer is a second non-ionic polymer such as a linear poly(vinyl pyrrolidone). “Linear poly(vinyl pyrrolidone)” as used herein specifically excludes any cross-linked polyvinylpyrrolidone, such as KOLLIDON® CL and KOLLIDON® CL-10 (BASF) and POLYPLASDONE® XL and POLYPLASDONE® XL-10 (Ashland). In certain embodiments, the linear poly(vinyl pyrrolidone) can have an M_{w} between about 200 kDa and 2 MDa; or between about 200 kDa and 1.75 MDa; between about 1 MDa and 1.7 MDa. Examples of commercial linear poly(vinyl pyrrolidone) include those from Ashland LLC (Covington, Ky.) or Ashland Specialty Ingredients (Wilmington, Del.) such as, PVP K-60 (M_{W} 240 kDa-470 kDa) and PVP K-90 (M_{W} 1 MDa-1.7 MDa).

When present, the optional excipients can be selected from the group consisting of a binder, a diluent, a glidant, a lubricant, a coloring agent, a coating agent, and mixtures thereof. Examples of suitable binders, glidants, lubricants, coating agents, and coloring agents are described further below.

In certain embodiments, the core can comprise about 0.1 wt % to about 20 wt % of excipients. In another embodiment, the core can comprise about 0.1 wt % to about 15 wt % of the excipients. In another embodiment, the core can comprise less than about 10 wt % excipients (e.g., about 0.1 wt % to about 10 wt %).

Binders include pharmaceutically acceptable agents can hold various ingredients together in a cohesive mix, for example, to hold together an active pharmaceutical ingredient and inactive ingredients. Examples of suitable binders include, but are not limited to, dry binders such as partially pre-gelatinized starch (e.g., UNI-PURE® DW partially pre-gelatinized maize starch, National Starch & Chemical); anhydrous lactose, and dibasic calcium phosphate dehydrate; and wet binders such as povidone, methyl cellulose, ethyl cellulose, hydroxypropyl cellulose, carboxymethyl cellulose, hydroxypropyl methyl cellulose, and mixtures thereof.

Diluents include in the core can include pharmaceutically acceptable inert fillers. Suitable diluents include, but are not limited to lactose, starch, dibasic calcium phosphate, saccharides, mannitol; Pearlitol® SD 200 (Roquette Freres SA, Lestrem, France); starch; sorbitol; sucrose; glucose and/or mixtures of the foregoing.

Lubricants include pharmaceutically acceptable agents that can prevent ingredients from clumping together and/or from sticking to certain processing equipment, such as tablet punches or capsule filling machines. Suitable lubricants include for example, talc, stearic acid, magnesium stearate, calcium stearate, and sodium stearyl fumarate.

Glidants include pharmaceutically acceptable agents that can promote powder flow by reducing interparticle friction and cohesion. Suitable glidants include, for example, colloidal silicon dioxide such as Aerosil® 200 (a hydrophilic fumed silica with a specific surface area of 200 m²/g; Evonik Corp., Piscataway, N.J.) or CAB-O-SIL, M-5P (a fumed silica with a specific surface area of 200 m²/g; Cabot Corp., Billerica, Mass.), talc, and magnesium carbonate.

The optional film coating agents used herein may include lactose, hydroxypropyl methylcellulose, triacetin, titanium dioxide, polyvinyl alcohol, talc, lecithin, sodium alginate, stearic acid, glyceride, oils and gelatins, sugar derivatives, polyethylene glycol, and combinations thereof.

Coloring agent as used herein include, but are not limited to, pharmaceutically acceptable dyes, such as FD&C dyes including Blue No. 1, Blue No. 1 Lake, Blue No. 1—Aluminum Lake, Blue No. 2, Blue No. 2—Aluminum Lake, Green No. 3, Red No. 3, Red No. 40, Red No. 40—Aluminum Lake, Yellow No. 5, Yellow No. 5—Aluminum Lake, Yellow No. 6, and Yellow No. 6-Aluminum Lake; and inorganic colorants, such as alumina, titanium dioxide, ferric oxide brown, ferric oxide orange, ferric oxide red, ferric oxide yellow, ferrosoferric oxide, ferrous oxide; and natural colorants such as caramel and annatto extract.

In certain embodiments of any of the cores described above, the one or more optional excipients comprises a lubricant and/or a glidant. For example, for of any of the cores described above, the one or more optional excipients comprises magnesium stearate and/or silicon dioxide.

In another embodiment, the core can comprise or consist essentially of the API, the matrix forming agent, the extended-release agent, the wicking agent, and a diluent and/or lubricant. For example, the core can comprise or consist essentially of:

about 5-40 wt % of the API (or about 5-30 wt %);

about 55-90 wt % of the sum of the matrix forming agent and extended-release agents; and

about 5-40 wt % of the wicking agent (or about 15-35 wt %).

In another embodiment, the core can comprise or consist essentially of essentially of the API, the matrix forming agent, the extended-release agent, the wicking agent, and a diluent and/or lubricant. For example, the core can comprise or consist essentially of:

about 5-40 wt % of the API (or about 5-30 wt %);

about 20-30 wt % of the matrix forming agents;

about 20-40 wt % of the extended-release agents; and

about 5-40 wt % of the wicking agent (or about 15-35 wt %).

In another embodiment, the core can comprise or consist essentially of the API, the matrix forming agent, the extended-release agent, the wicking agent, and a diluent and/or lubricant. For example, the core can comprise or consist essentially of:

about 5-30 wt % of the API;

about 20-30 wt % of the matrix forming agents;

about 25-35 wt % of the extended-release agents; and

about 15-35 wt % of the wicking agent.

In another embodiment, when the core does not comprise the wicking agent (e.g., the core consists essentially of the API, the matrix forming agent, the extended-release agent, and a diluent and/or lubricant). For example, the core can comprise or consist essentially of:

about 5-45 wt % of the API (or about 5-35 wt %); and

about 55-95 wt % of the sum of the matrix forming agent and extended-release agents.

In another embodiment, when the core does not comprise the wicking agent (e.g., the core consists essentially of the API, the matrix forming agent, the extended-release agent, and a diluent and/or lubricant), then the core can comprise or consist essentially of:

about 5-45 wt % of the API (or about 5-35 wt %);

about 20-50 wt % of the matrix forming agent; and

about 30-60 wt % of the one or more extended-release agents.

In another embodiment, when the core does not comprise the wicking agent (e.g., the core consists essentially of the API, the matrix forming agent, the extended-release agent, and a diluent and/or lubricant), then the core can comprise or consist essentially of:

about 5-35 wt % of the API (or about 10-40 wt %);

about 20-30 wt % of the matrix forming agent; and

about 30-60 wt % of the one or more extended-release agents.

Preparation of the Core

The dosage core can be prepared according to any methods familiar to those skilled in the art. In one embodiment, the core can be prepared by combining an API with one or more extended-release agents, and a wicking agent, when present, to provide a first blend. The combining may be blending the components in a suitable blender, such as a “V” blender. In other examples, the combining may further include optional roll compacting the first blend.

The first blend, whether compacted or not, may be processed to provide particle size reduction, for example, by passing the first blend through a Fitzmill equipped with a suitable sized screen, such as a #0 screen, and at a suitable speed, such as a “low”, “medium” or “high” rotor speed as is familiar to those skilled in the art. In other examples, the first blend is passed through one or more screens to provide a screened first blend. In certain embodiments, the first blend is passed through a Fitzmill equipped with a #0 screen, and at a “medium” rotor speed. In such examples, the first blend is processed to provide a milled first blend.

The first blend, screened first blend, or milled first blend may be combined with one or more optional excipients to provide a final blend. In one example, the optional excipients include a lubricant and/or a glidant. For example, the one or more optional excipients may comprise magnesium stearate and/or silicon dioxide. In another example, the one or more optional excipients may comprise magnesium stearate. The combining may be blending the components in a suitable blender, such as a “V” blender. The final blend may be processed to provide particle size reduction and/or uniformity, for example, by passing the final blend through a Fitzmill or through one or more screens to provide a screened final blend.

A portion of the final blend, screened final blend, or milled final blend may be compressed to provide a compressed tablet, wherein the portion of the final blend comprises a therapeutically effective amount of the API. Such compressed tablets may be coated with a coating layer, as described below.

In another embodiment, a portion of the final blend, screened final blend, or milled final blend may be compressed to provide a compressed minitablet, wherein the portion of the final blend comprises less than a therapeutically effective amount of the API. Such compressed minitablets may be coated with a coating layer, as described below. A plurality of minitablets or coated minitablets may be filled into a capsule shell, wherein the plurality of compressed minitablets comprises a therapeutically effective amount of the API.

Alternatively, a portion of preceding final blend, screened final blend, or milled final blend comprising a therapeutically effective amount of the API may be filled into a hard gelatin capsule to provide an extended-release capsule formulation.

In certain embodiments, the oral dosage forms, herein can have a total mass, including core and any coating layers, of about 200 to about 2,000 mg; or about 300 to 1,500 mg, or about 350 to 1000 mg. For example, where the oral dosage form comprises 82.5 mg, or 165 mg, or 330 mg of pregabalin, then the total mass of the dosage form can be about 500 mg to about 1,500 mg; or about 500 mg to about 1200 mg; or about 750 mg to about 1200 mg; or about 850 mg to about 1200 mg; or about 850 mg to about 950 mg; or about 900 mg to about 1200 mg; or about 1000 mg to about 1200 mg; or about 1100 mg to about 1200 mg; or about 800 mg to about 1000 mg; or about 900 mg to about 1000 mg. In certain embodiments, the oral dosage form comprises 82.5 mg, or 165 mg, or 330 mg of pregabalin, the core comprises the wicking agent, and the total mass of the dosage form is about 1000 mg to about 1200 mg; or about 1100 mg to about 1200 mg. In certain other embodiments, the oral dosage form comprises 82.5 mg, or 165 mg, or 330 mg of pregabalin, the core does not comprise the wicking agent. and the total mass of the dosage form is about 800 mg to about 1000 mg; or about 900 mg to about 1000 mg.

Optional Coating Layer

In certain embodiments, the extended-release formulation comprises the core and the optional coating layer formed over the core (i.e., have a “coating layer” formed over the core). The coating layer, when applied, may be applied to the core by methods familiar to those skilled in the art.

The film formers used for the coating process may, for example, be cellulose derivatives such as methyl cellulose (MC), ethyl cellulose (EC), hydroxyethyl cellulose (HEC), methacrylic acid/acrylate copolymers, HPMC, vinyl polymers, or natural film formers, such as shellac. Examples of commercially available film formers include, but are not limited to, Opadry® (HPMC), Opadry® II (poly(vinyl alcohol)), and Surelease® (Ethylcellulose Dispersion Type B NF) Film Coating Systems (each available from Colorcon, Inc., North Wales, Pa.), PHARMACOAT 606 HPMC (low viscosity, about 6 cP for a 2% solution in water at 20° C. by the USP method, 29% methoxy, 9% hydroxypropoxy substitution; Shin-Etsu Chemical Co., Ltd., Tokyo, Japan); ETHOCEL Standard 45 Premium ethylcellulose (Ubbelohde Viscosity of about 41-49 cP, 48-49.5 wt % ethoxyl content; Dow Chemical, Midland, Mich.), and mixtures thereof.

In one embodiment, the extended-release formulation comprises the core and a coating layer formed over the core, wherein the coating layer comprises a cellulose derivative, a methacrylic acid/acrylate copolymers, a vinyl polymer, a natural film former, or a mixture thereof.

In another embodiment, the coating layer comprises a methyl cellulose, a ethyl cellulose, a hydroxyethyl cellulose, a methacrylic acid/acrylate copolymer, a HPMC, a poly(vinyl alcohol, or a mixture thereof. In another embodiment, the coating layer comprises a HPMC, a poly(vinyl alcohol), or a mixture thereof.

In another embodiment, the coating layer comprises a HPMC, an ethylcellulose, or a mixture thereof.

In another embodiment, the coating layer comprises a mixture of a HPMC and a ethylcellulose.

For example, for any of the preceding embodiment where the core comprises (a) a polyethylene oxide (PEO) and a hydroxyalkylcellulose (HAC), the coating layer can comprise a methyl cellulose, a ethyl cellulose, a hydroxyethyl cellulose, a methacrylic acid/acrylate copolymer, a HPMC, a poly(vinyl alcohol), or a mixture thereof. In a particular embodiment, the coating layer can comprise a HPMC, a poly(vinyl alcohol), or a mixture thereof. In a particular embodiment, the coating layer can comprise a HPMC, such as an Opadry® coating. In a particular embodiment, the coating layer can comprise a poly(vinyl alcohol), such as an Opadry II® coating (for example, Opadry II (85F18422 white) contains poly(vinyl alcohol) and PEG 3350). The coating can be applied to achieve a desired increase in mass of the core. For example, the coating can be applied to increase the mass of the core by about 2-5 wt %; or about 2-4 wt %; or about 2-3 wt %; or about 2.3-2.8 wt %.

For example, for any of the preceding embodiment where the core comprises (b) a hydroxyalkyl alkylcellulose (HAAC) and a carboxymethylcellulose or salt thereof, then the coating layer can comprises a methyl cellulose, a ethyl cellulose, a hydroxyethyl cellulose, a methacrylic acid/acrylate copolymer, a HPMC, a poly(vinyl alcohol, or a mixture thereof. In a particular embodiment, the coating layer can comprise a HPMC, an ethylcellulose, or a mixture thereof. In another particular embodiment, the coating layer comprises a mixture of a HPMC and a ethylcellulose, such as an Opadry® (HPMC) coating and a Surelease® (Ethylcellulose Dispersion Type B NF) coating. When the coating layer comprises a mixture of film formers, then the coating layer, itself, can be a mixture of the recited firm formers or the coating layer can comprise a first layer formed over the core and a second layer formed over the first layer, where the first and second layers are a first film former (e.g., HPMC) and second film formers (e.g., ethylcellulose), respectively. Alternatively, the first film former can comprise ethylcellulose and second film formers can comprise HPMC.

When a mixture of two film formers are used, the can be present at a weight ratio of about 2:1 to about 1:2; or about 1.5:1 to 1:1. In one example, first film former is a HPMC and second film former is an ethylcellulose, and weight ratio of HPMC to ethylcellulose is about 2:1 to about 1:2; or about 1.5:1 to 1:1. Such a coating can be applied to achieve a desired increase in mass of the core. For example, the coating can be applied to increase the mass of the core by about 2-7 wt %; or about 3-5 wt %; or about 3-4 wt %.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### EXAMPLES

### Examples 1-3

| | Ex. 1 | | Ex. 2 | | Ex. 3 | |
|---|---|---|---|---|---|---|
| Ingred./Form. # | [mg/tablet] | [%, w/w] | [mg/tablet] | [%, w/w] | [mg/tablet] | [%, w/w] |
| Core | | | | | | |
| PART I | | | | | | |
| pregabalin | 330.0 | 27.97 | 165.0 | 14.05 | 82.5 | 7.03 |
| Kollidon ® SR | 301.0 | 25.51 | 320.0 | 27.26 | 320.0 | 27.26 |
| Microcrystalline | 197.5 | 16.74 | 320.25 | 27.28 | 342.75 | 29.20 |
| Cellulose | | | | | | |
| Polyethylene | 260.0 | 22.03 | 280.0 | 23.85 | 340.0 | 28.96 |
| oxide | | | | | | |
| Carbopol ®71G | 55.9 | 4.74 | 59.0 | 5.0 | 59.0 | 5.0 |
| Total ER agents | | 26.77 | | 28.85 | | 33.96 |
| PART II | | | | | | |
| magnesium | 5.6 | 0.47 | 5.75 | 0.5 | 5.75 | 0.5 |
| stearate | | | | | | |
| Core Total | 1150.0 | 100.0 | 1150.0 | 100.0 | 1150.0 | 100.0 |
| Coating Layer | | | | | | |
| Solids | 30.0 | 2.56 | 24.0 | 2.04 | 24.0 | 2.04 |
| contribution | | | | | | |
| from Opadry II | | | | | | |
| Tablet total | 1180.0 | 100.0 | 1174.0 | 100.0 | 1174.0 | 100.0 |

Polyethylene oxide = WSR N60K
Microcrystalline Cellulose = Avicel ® PH 102
Opadry II = White Opadry II (85F18422) 20% w/w coating suspension

The Part I pregabalin and a portion of the Part I microcrystalline cellulose were blended in a suitably sized V blender. The Part I Kollidon®SR, Carbopol®71G, polyethylene oxide, and remaining microcrystalline cellulose were added to the preceding blend and further blended. After discharging the blend, the material was passed through a Fitzmill equipped with a #0 screen size, blades position at knives forward on medium speed to form a Part I milled material. The Part II magnesium stearate was passed through a #18 mesh screen and blended with the Part I milled material in a suitably sized V blender to provide the final blended material. The final blended material was compressed into tablets in a rotary tablet press. The compressed tablets were coated with the OPADRY II coating suspension in suitably sized perforated coating pan. After the specified weight gain was been achieved, the tablets were dried. Examples 2-3 were prepared in the same manner as Example 1.

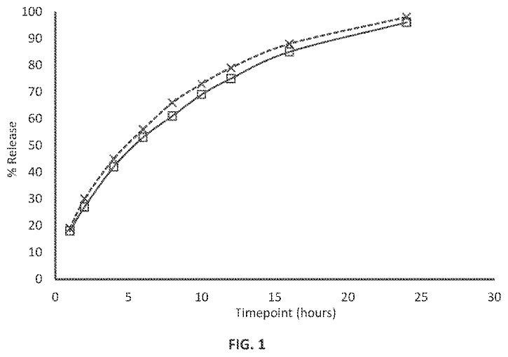

Dissolution of each Examples 1-3 were conducted using the US FDA Method (0.06N HCl, App. II @ 50 rpm) or in a pH 4.5 acetate buffer, App II at 50 rpm, and compared to the equivalent dosage strength of the Lyrica® CR product (Pfizer, NY, N.Y.). Results of the dissolution studies are shown in FIGS. 1 (0.06N HCl, 330 mg), 2 (acetate buffer, 330 mg), 3 (acetate buffer, 165 mg) and 4 (acetate buffer, 82.5 mg).

### Examples 4-6

| | Ex. 4 | | Ex. 5 | | Ex. 6 | |
|---|---|---|---|---|---|---|
| Ingred./Form. # | [mg/tablet] | [%, w/w] | [mg/tablet] | [%, w/w] | [mg/tablet] | [%, w/w] |
| Core | | | | | | |
| PART I | | | | | | |
| pregabalin | 330.0 | 34.7 | 165.0 | 17.4 | 82.5 | 8.7 |
| Kollidon ® SR | 281.8 | 29.6 | 360.5 | 37.9 | 399.9 | 42.0 |
| Polyethylene | 247.2 | 26.0 | 316.2 | 33.2 | 350.8 | 36.9 |
| oxide | | | | | | |
| Carbopol ®71G | 61.8 | 6.5 | 79.1 | 8.3 | 87.7 | 9.2 |
| Total ER agents | | 32.5 | | 41.5 | | 46.1 |
| PART II | | | | | | |
| magnesium | 6.2 | 0.7 | 6.2 | 0.7 | 6.2 | 0.7 |
| stearate | | | | | | |
| Core Total | 927.0 | 97.5 | 927.0 | 97.5 | 927.0 | 97.5 |
| Coating Layer | | | | | | |
| Solids | 24.0 | 2.5 | 24.0 | 2.5 | 24.0 | 2.5 |
| contribution | | | | | | |
| from Opadry II | | | | | | |
| Tablet total | 951.0 | 100.0 | 951.0 | 100.0 | 951.0 | 100.0 |

Polyethylene oxide = WSR N60K
Opadry II = White Opadry II (85F18422) 20% w/w coating suspension

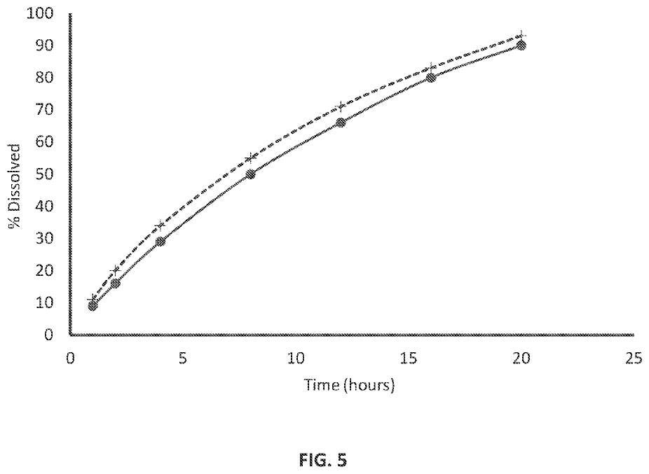

Examples 4-6 were prepared in the same manner as Examples 1-3, but omitting the microcrystalline cellulose. Dissolution of each Examples 4-6 were conducted water, App II at 150 rpm and compared to the equivalent dosage strength of the Lyrica® CR product (Pfizer, NY, N.Y.). Results of the dissolution studies are shown in FIGS. 5 (330 mg), 6 (165 mg), and 7 (82.5 mg).

### Example 7

| | Ex. 7 | |
|---|---|---|
| Ingred./Form. # | [mg/tablet] | [%, w/w] |
| Core | | |
| PART I | | |
| pregabalin | 330.0 | 26.7 |
| Kollidon ® SR | 281.8 | 22.8 |
| Povidone K90 | 309.0 | 25.0 |
| Polyethylene oxide WSR N60K | 247.2 | 20.0 |
| Carbopol ® 71G | 61.8 | 5.0 |
| Total ER agents | | 50.0 |
| PART II | | |
| magnesium stearate | 6.2 | 0.5 |
| Core Total | 1236.0 | 100.0 |

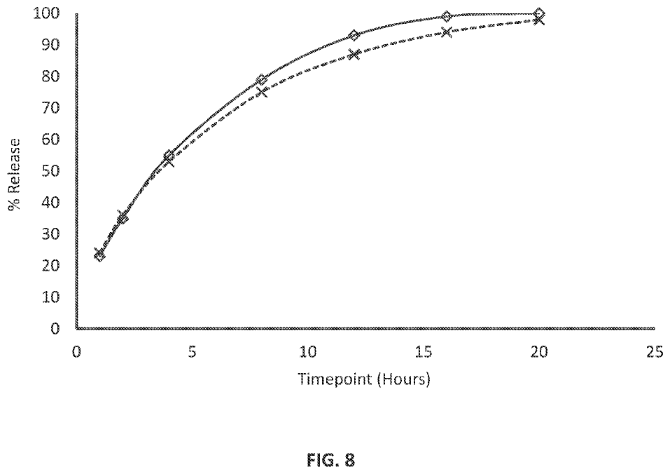

Example 7 was prepared in the same manner as Examples 1-3, substituting Povidone K90 for the microcrystalline cellulose, but without the addition of a coating layer. Dissolution of the tablets of Example 7 were conducted water, App II at 150 rpm and compared to the equivalent dosage strength of the Lyrica® CR product (Pfizer, NY, NY). Results of the dissolution studies are shown in FIGS. 8 (330 mg).

1. An extended-release formulation comprising a core and an optional coating layer surrounding the core, wherein the core comprises:
a therapeutically effective amount of an active pharmaceutical ingredient (API) that is pregabalin or a pharmaceutically acceptable salt or solvate thereof;
a non-swelling matrix forming agent, wherein the non-swelling matrix forming agent comprises a water-soluble agent and a water-insoluble polymer;
one or more extended-release agents;
an optional non-swelling wicking agent; and
one or more optional excipients.

2. The formulation of claim 1, wherein the non-swelling matrix forming agent comprises
a water-soluble agent that is a poly(vinyl pyrrolidone), a polyethylene glycol, or a mixture thereof; and
a water-insoluble polymer that is a poly(vinyl acetate), a polyacrylic acid, cellulose acetate, ethyl cellulose, or a mixture thereof.

3. The formulation of claim 1, wherein the non-swelling matrix forming agent comprises poly(vinyl pyrrolidone) and poly(vinyl acetate).

4. The formulation of claim 1, wherein the extended-release agent comprises a non-ionic polymer and/or an ionic polymer, wherein
the ionic polymer is a poly(acrylic acid), a carbomer, alginic acid, carrageenan, xanthan gum, carrageenan, or mixtures thereof; and
the non-ionic polymer is a polyethylene oxide, a polysaccharide or a mixture thereof.

5. The formulation of claim 4, wherein the extended-release agents comprise
(a) a poly(acrylic acid) or a carbomer and
(b) a polyethylene oxide.

6. The formulation of claim 5, wherein the extended-release agents further comprise a linear poly(vinyl pyrrolidone).

7. The formulation of claim 1, wherein the non-swelling wicking agent is a microcrystalline cellulose, sodium lauryl sulfate, colloidal silicon dioxide, low molecular weight polyvinylpyrrolidone, or a mixture thereof.

8. The formulation of claim 7, wherein the non-swelling wicking agent is a microcrystalline cellulose or silicified microcrystalline cellulose.

9. The formulation of claim 1, wherein the core comprises,
about 5-40 wt % of the API;
about 55-90 wt % of the sum of the non-swelling matrix forming agent and the one or more extended-release agents; and
about 5-40 wt % of the non-swelling wicking agent.

10. The formulation of claim 1, wherein the core comprises,
about 5-40 wt % of the API;
about 20-30 wt % of the matrix forming agents;
about 20-40 wt % of the extended-release agents; and
about 5-40 wt % of the wicking agent.

11. The formulation of claim 9, wherein the core comprises about 15-35 wt % of the non-swelling wicking agent.

12. The formulation of claim 1, wherein the core does not comprise the wicking agent.

13. The formulation of claim 12, wherein the core consists essentially of the API, the matrix forming agent, the one or more extended-release agents, and a diluent and/or lubricant.

14. The formulation of claim 12, wherein the core consists essentially of,
about 5-45 wt % of the API;
about 20-50 wt % of the matrix forming agent; and
about 30-60 wt % of the one or more extended-release agents.

15. The formulation of claim 1, in the form of a modified-release tablet.

16. A process for preparing the formulation of claim 1, comprising combining the API, the non-swelling matrix forming agent; the one or more extended-release agents; and optionally the non-swelling wicking agent to provide a first blend; and combining the first blend with one or more optional excipients to provide a final blend.

17. The process of claim 16, further comprising compressing a portion of the final blend to provide the core, wherein the portion of the final blend comprises a therapeutically effective amount of the API.

18. The process of claim 17, further comprising forming a coating layer over the core.

19. A method for treating a disease or condition selected from the group consisting of neuropathic pain associated with diabetic peripheral neuropathy (DPN), postherpetic neuralgia (PHN), seizure disorder, anxiety, alcohol use disorder, fibromyalgia, cancer pain, post-operative pain, restless legs syndrome, and nerve pain due to spinal cord injury in a patient in need of such treatment comprising administering to the patient a formulation according to claim 1, wherein the administering is once-daily administration.

20. The method of claim 19, wherein the disease or condition is neuropathic pain associated with diabetic peripheral neuropathy (DPN) or postherpetic neuralgia (PHN).
